(19) United States
(12) Patent Application Publication
(10) Pub. No.: US 2025/0164479 A1
(43) Pub. Date: May 22, 2025

(54) ANALYTICAL METHOD

(71) Applicant: ATTANA AB, Sollentuna (SE)

(72) Inventor: Daniel WALLINDER, Sollentuna (SE)

(21) Appl. No.: 18/837,333

(22) PCT Filed: Feb. 8, 2023
(86) PCT No.: PCT/EP2023/053127

§ 371 Date: Aug. 9, 2024

(30) Foreign Application Priority Data
Feb. 11, 2022 (GB) 2201800.6
Dec. 13, 2022 (GB) 2218773.6

(51) Int. Cl. G01N 33/557; G01N 33/569

(52) U.S. Cl. CPC G01N 33/557; G01N 33/56983; G01N 2333/165; G01N 2469/20

(57) ABSTRACT

A method for assessing the neutralising potential of antibodies in a complex biological sample, the method comprising contacting the complex biological sample with a flow-based analytical device, the analytical device being provided with an antigen of interest, allowing antibodies present in the complex biological sample to interact with the antigen of interest, and determining the association-rate binding constant ka of the antibody-antigen interaction and/or the dissociation-rate binding constant ka of the anti-body-antigen interaction.

This invention relates to analytical methods. In particular, though not exclusively, it relates to methods for assessing the neutralising potential of antibodies in biological samples.

A variety of solution phase and solid phase techniques are available for measuring antibodies in biological samples, most commonly blood or serum (e.g. human blood or serum). However, current methods typically only detect, or at best semi quantify, the titres of human immune antibodies. This measurement merely indicates that the antibodies present are capable of binding to the antigen of interest, but provides essentially no information about the strength or other qualities of the antibody-antigen interaction. In addition, existing techniques suffer from a significant lack of standardization, with assays for antibodies against different pathogens (e.g. neutralizing assays against viruses or bacteria, for example) requiring different preparative and analytical conditions.

Measurement of the kinetics of antibody-antigen interactions can be undertaken using a variety of techniques. However, such techniques have hitherto measured the interactions in buffer solutions, rather than biological media, due to the interference caused by other components present in biological media. Brien et al. (J. Virol. 2013, 87 (13): 7747-7753) reports the determination of kinetic parameters in the design of antibodies against Dengue virus. However, the analysis is conducted using buffer solution. US 2020/0033264 describes a surface plasmon resonance (SPR)-based analysis of antibody concentrations in samples derived from biological samples. However, SPR and other evanescent wave-based techniques are adversely affected by biological media, and hence this approach requires prior treatment of the sample, such as heat treatment, dithiothreitol, dialysis, concentration and/or purification of the immunoglobulin G (IgG) component of the sample. The samples analysed are thus not complex biological samples in the sense of containing all soluble components originally present in the sample. Accordingly, the interaction kinetics determined under such conditions may not fully reflect the interaction kinetics which would apply in vivo.

WO 2021/026251 describes a method for determining the kinetics of a binding interaction between first and second biomolecules. However, the second biomolecule is required to be labelled with a magnetic nanoparticle in order to be detected. In addition, although samples are described as being derived from blood (and other biological fluids), these are mixed with surfactants etc. to make them suitable for analysis. The presence of magnetic nanoparticles and/or surfactants will affect the accurate determination of interaction kinetics, and is thus undesirable.

Yue Gu et al. https://doi.org/10.1101/2022.03.06.22271809 (pre-print, March 2022) discloses a biosensor-based study of antibody binding to antigens of interest. However, no connection is made between the kinetics of the antibody-antigen interaction and the neutralising capacity of the antibody samples studied.

It has been found that total specific antibody concentration and total specific IgG concentration in a sample are not wholly predictive of the immunological status of the subject from whom the sample was obtained, especially without the use of complex biological samples as defined above. Immunological capability relies on the ‘quality’ of the antibody-antigen interaction, such that antibodies present in a subject are able to neutralise a pathogen (e.g. prevent the pathogen from reproducing or from entering cells). A need therefore exists for a method for more accurately assessing the neutralising capability of antibodies in complex biological samples.

According to a first aspect of the invention, there is provided a method for assessing the neutralising potential (or capacity) of antibodies in a complex biological sample, the method comprising introducing the complex biological sample into a flow-based analytical device, the analytical device being provided with an antigen of interest, allowing antibodies present in the complex biological sample to interact with the antigen of interest, and determining the association-rate binding constant kₐ of the antibody-antigen interaction and/or the dissociation-rate binding constant k_{d} of the antibody-antigen interaction.

In the method of the invention, the association rate-binding constant kₐ (also sometimes referred to as the on-rate binding constant (kₒₙ)) of the antibody-antigen interaction is used to assess the neutralising capacity of the antibodies in a complex biological sample obtained from a subject. It has been surprisingly found by the inventors that this assessment is predictive of the functional immune status of the subject. The relationship between kₐ of the antibodies of a complex biological sample in their interaction with an antigen of interest, and the neutralising capacity or potential of those antibodies, has not been studied or recognised previously. According to the present invention, the higher the kₐ of the interaction, the greater in general the neutralising capacity of the antibodies in the sample.

Alternatively or in addition, the dissociation-rate binding constant k_{d} (also sometimes referred to as the off-rate binding constant (k_{off})) of the antibody-antigen interaction is determined. The k_{d} is determined following cessation of introduction of the complex biological sample into the analytical device, i.e. such that the complex biological sample is rapidly replaced in the flow-based analytical device by media which does not interact with the antigen of interest, e.g. buffer solution. During this step, weakly-interacting components of the complex biological sample tend to rapidly dissociate from the antigen, whilst strongly-interacting components such as specific antibodies tend to dissociate more slowly. It has been surprisingly found by the inventors that this assessment is also predictive of the functional immune status of the subject. According to the present invention, the lower the k_{d}, the greater in general the neutralising capacity of the antibodies in the sample.

The skilled person would be aware of how to determine the kinetic parameters mentioned herein. For example, determination of kinetic parameters using e.g. biosensor approaches have been described by Brien et al. (J. Virol. 2013, 87 (13): 7747-7753); Myszka (J. Molec. Recognit., 1999, 12:279; or Curr. Opin. Biotechnol. 1997, 8 (1): 50-57); and Morton and Myszka (Methods Enzymol., 1998, 295:268-294). The contents of these documents are incorporated by reference herein in their entirety, and in particular the teaching of these documents relating to determining kinetic parameters of antibody-antigen interactions.

The methods of the present invention allow significant standardization of testing protocols between different antigens of interest (and hence different pathogens, cell types etc). It is well known that existing assays based on binding interactions between biological samples and antigens of interest from different sources require, for example, different incubation times and different incubation conditions, since the interactions involved can vary from strong, specific and fast to weak, less specific and slow. The methods of the present invention allow a wide range of assays based on different sources of antigen to be conducted under very similar conditions, since the interaction kinetics can be measured in a similar manner in each instance. For similar reasons, the methods of the invention allow for improved reproducibility compared to known methods. When samples from the same source are tested using different known techniques, variations in results (e.g. total Ab titre, total specific Ab titre) are observed, for example because of different sample preparation methods, incubation times and incubation conditions. The methods of the invention avoid these problems. The interaction kinetics are an intrinsic property of the binding species involved, and by measuring these in a complex biological sample with minimal alteration thereof, accuracy and reproducibility are preserved. In addition, at least some of the kinetic parameters can be accurately determined in short periods of time, which allows useful results to be obtained from the methods of the invention in a time-span which is shorter than prior art methods.

In carrying out the methods of the present invention the ‘antigen of interest’ (sometimes referred to simply as ‘the antigen’) may be in the form of an isolated molecular species (e.g. peptide, protein, protein fragment, polynucleotide, polysaccharide), or may be in the form of a partial or whole virus, bacterium, other microorganism or cell (e.g. tumor cell) which contains or expresses that molecular species.

In a preferred embodiment, the binding of all soluble components of the complex biological sample to the antigen of interest is also determined. Such components include antibodies of all isotypes which are capable of recognising and binding to the antigen of interest, together with proteins (including antibodies directed to other antigens) and other components of the complex biological sample (e.g. serum proteins such as albumin, fibrinogen etc.) which become attached during the interaction phase by means of weak chemical interactions (such as van der Waal's forces). The binding of all such components is representative of the total (that is, specific (higher affinity) and non-specific (lower affinity)) immunological capacity of the complex biological sample. It should be understood that even soluble components of the complex biological sample which only have predominantly weak interactions with the antigen can contribute to the overall neutralising capacity of the sample, particularly if they are present in relatively high concentrations. The measurement of the overall binding of soluble components from the sample thus provides this useful additional information.

Similarly, it is preferred that the titre of specific antibody binding to the antigen of interest is also determined. The term ‘specific antibody’ in this context refers to antibodies of all isotypes which are capable of recognising and binding to the antigen of interest with high affinity (i.e. with a faster association rate to the antigen than the antigen to the target receptor/binding partner (e.g. in the case of SARS-COV-2 spike protein, the native binding partner is ACE2)). A slower dissociation rate compared to the antigen-receptor/binding partner dissociation rate will accordingly also be important in terms of ‘high affinity’. The titre of specific antibody is thus typically measured following washing of the analytical system, the washing step removing proteins (including antibodies directed to other antigens and/or antibodies having low affinity for the antigen of interest) and other components of the complex biological sample which become attached during the interaction phase by means of weak chemical interactions (such as van der Waal's forces). According to the present invention, the higher the titre of specific antibody in the complex biological sample, the greater the neutralising capacity of the sample.

In another preferred embodiment, the association-rate binding constant kₐ and the dissociation-rate binding constant k_{d} of the antibody-antigen interaction are determined.

In another preferred embodiment, the titre of specific IgG binding to the antigen of interest is also determined. IgG antibodies are the main antibodies circulating in blood, and hence a key component of humoral immunity. According to the present invention, the higher the specific IgG titre, the greater the neutralising capacity of the antibodies in the sample. Specific IgG titre can conveniently be determined after the dissociation phase by introducing an anti-IgG antibody into the analytical device.

In preferred embodiments, two or more of kₐ, all soluble component binding, specific antibody titre, k_{d}, and specific IgG titre are determined using the same analytical device. Preferably the analytical conditions (e.g. pH, temperature) are also the same for each determination. Such an approach ensures that the overall predictive capability of the method of the invention is as high as possible.

According to the invention, the combination of kₐ, specific antibody titre, k_{d}, and specific IgG titre is highly predictive of the neutralising potential of antibodies in a biological sample. By correlating such measured parameters with actual neutralising capability of samples from the subject (e.g. prevention of pathogen replication), or following in vivo challenge of the subject with a pathogen of interest, it is possible to rank the parameters in terms of relative importance in terms of neutralising potential for the pathogen of interest.

In certain embodiments, the complex biological sample is selected from blood, serum, plasma, saliva, sputum, biopsy eluate and lavage liquid. In preferred embodiments, the complex biological sample is selected from blood, serum and plasma. In a convenient embodiment, the blood, serum or plasma is derived from capillary blood, e.g. from a finger-prick sample (including dried samples of any of these types, e.g. pin-prick dried blood samples). This minimises the time and effort needed to obtain the sample, and makes the method of the invention highly suitable for large scale, rapid testing of subjects.

In preferred embodiments, the complex biological sample is used without removal of cells. This makes the method faster and more convenient, and yields more accurate results in terms of predictability of neutralising capacity of the sample. In preferred embodiments, no additional chemical or physical species are added to the sample which by themselves elicit a detectable signal in the analytical measurement mode used. Similarly, it is preferred that no chemical or physical species are added to the sample which alter the kinetics of the interactions between species already present in the sample. For example, the addition of a surfactant causes certain species in a sample to be separated (at least to a degree) from other species having a different hydrophile-lipophile balance. Equally, the addition of some chemical species can affect the tertiary structure of antibodies and/or antigens, by altering intra-and/or intermolecular hydrogen bonds, Van der Waal's forces, hydrophobic bonding etc. of the macromolecules. Under such conditions, the true kinetics of the interaction of interest can no longer be obtained from such a sample, since at least some of the components of the sample are no longer able to interact in their native manner. In some embodiments, the complex biological sample is diluted, e.g. with a suitable buffer. Dilution with a buffer prior to introduction of the complex biological sample into the analytical device allows the measured signal to be tailored to the analytical measurement mode being employed. However, soluble components of the complex biological sample (and preferably cells, in embodiments where the complex biological sample contains cells) are not removed by this process, unlike in certain prior art approaches. For highly sensitive analytical devices such as biosensors, dilution of the sample is preferred, and the running buffer of the biosensor is conveniently employed in such instances. Depending on the nature of the complex biological sample, dilutions may range from 1 in 2 to 1 in 1000, for example. A typical dilution of a blood, serum or plasma sample may be from 1 in 2 to 1 in 500, such as 1 in 10 to 1 in 200, 1 in 10 to 1 in 100, or 1 in 10 to 1 in 50. As will be appreciated by the skilled person, more accurate determination of kₐ and k_{d} may be obtained by conducting the analysis at multiple concentrations of the complex biological sample. Dilution with a buffer (e.g. running buffer) is employed for this purpose.

Where the complex biological sample is blood, plasma or serum (in particular, blood or plasma), an anticoagulant may be added to the sample to prevent clotting of the sample prior to or during the analysis. A divalent ion (e.g. Ca²⁺, Mg²) chelating agent such as EDTA is conveniently employed.

In embodiments, kₐ is determined over a time period which ends prior to the maximum level of antibody-antigen interaction being reached, such as the time period which ends at the point where 50% of the maximum level of antibody-antigen interaction is reached. In a typical embodiment of the method of the invention, upon introduction of the complex biological sample into the analytical device an association phase takes place. As described above, the association phase includes the potential interaction of all components of the biological sample which are capable of binding to the antigen of interest, including high affinity antibodies, lower affinity antibodies and other components (e.g. proteins) of the biological sample. During this phase, a maximum level of binding is reached, which typically corresponds to an approximate plateauing of the measured signal in the analytical device (although, in practice, it is not necessary for the purposes of the present invention to reach a plateau per se). In typical embodiments, the association phase may be biphasic, with primarily fast, high kₐ interactions taking place in the initial period (when the rate of increase of the measured signal is relatively high), and with primarily slower interactions taking place in the later period (when the rate of increase of the measured signal is relatively low). By measuring the kₐ of the antibodies in the biological sample during the earlier part of the association phase, the measured kₐ more accurately reflects the kₐ of the highest affinity antibodies in the sample. Accordingly, in embodiments, kₐ may be determined during the first 10%, 20%, 30%, 40%, 50%, 60%, 70%, 80% or 90% of the observed association phase, with a preference for embodiments at the lower end of this range.

Similarly, as described above, following cessation of introduction of the complex biological sample into the analytical device, i.e. such that the complex biological sample is rapidly replaced in the flow-based analytical device by media which does not interact with the antigen of interest, e.g. buffer solution, a dissociation phase takes place. During this phase, weakly-interacting components of the complex biological sample rapidly dissociate from the antigen, whilst strongly-interacting components such as specific (i.e. high affinity) antibodies typically dissociate more slowly. In typical embodiments, therefore, the dissociation phase is also biphasic. By measuring the k_{d} of the antibodies in the biological sample during the later part of the dissociation phase (when the rate of decrease of the measured signal is relatively low), the measured k_{d} more accurately reflects the k_{d} of the slowest dissociating antibodies in the sample. Accordingly, in embodiments, k_{d} may be determined during the final 10%, 20%, 30%, 40%, 50%, 60%, 70%, 80% or 90% of the observed association phase, with a preference for embodiments at the lower (i.e. later in the dissociation phase) end of this range.

In certain embodiments, kₐ and/or k_{d} of the antibody-antigen interaction are compared with the equivalent rate constant(s) for the interaction between antibodies in a control complex biological sample from a subject demonstrating clinically significant immunity against a pathogen associated with the antigen (i.e. a pathogen, e.g. a virus, bacterium or other microorganism or cell (e.g. tumor cell), which expresses the antigen in such a manner that it is accessible to the immune system), or are compared with the equivalent rate constant(s) for the interaction between one or more commercially available therapeutic antibodies and their respective biological target antigens. The demonstration of clinically significant immunity against a pathogen may be by means of in vivo challenge, and may, for example, take place using a subject vaccinated against the pathogen. Clinical significance will vary from one pathogen/pathology to another, with complete absence of symptoms and/or an absence of detected pathogen in a sample taken from the subject not necessarily being required for this condition to be met. The skilled person would readily be able to ascertain the kinetic rate constants of commercially available therapeutic antibodies from published sources. Commercially available therapeutic antibodies may have, for example, kₐ in the range of 10³ to 10⁸, such as 10⁴ to 10⁷ M⁻¹s⁻¹ (e.g. 10⁴ to 10⁶) M⁻¹s⁻¹, and may have, for example, k_{d} in the range of 10⁻⁹ to 10⁻², such as 10⁻⁵ to 10⁻² (e.g. 10⁻⁵ to 10⁻³) s⁻¹. According to the invention, comparing the kₐ and/or k_{d} of the antibody-antigen interaction based on the complex biological sample with the corresponding kinetic parameters of commercially available therapeutic antibodies provides an indication, where one or both such parameters are comparable, of high neutralising potential of the antibodies in the biological sample.

In preferred embodiments, the flow-based analytical device is provided with a flow cell, the antigen of interest being immobilised in the flow cell and the complex biological sample being introduced into the flow cell for the purposes of the antibody-antigen interaction. Flow cells are widely used in analytical devices, and would be well known to the skilled person. They are particularly useful for kinetics analyses, since they allow the complex biological sample to be continuously provided to the antigen of interest (typically immobilised to a surface of the flow cell) at a constant concentration during the association phase (i.e. thereby preventing concentration/mass transfer effects on the measured kinetics). The flow cell should be made of biologically compatible material, preferably selected from, but not limited to, polyoxymethylene, polymethylmethacrylate, polyvinyl chloride and injection-moldable thermoplastics, such as polystyrene or acrylonitrile-butadiene-styrene. The dimensions of the flow cell should be suitable for determination of kinetic rate parameters for molecular interactions, i.e. the flow characteristics should allow for maintenance of the bulk solution concentration of the analytes contained in the complex biological sample at, or very close to, the surface of the flow cell provided with the antigen of interest, without substantial diffusion limitation of the analytes to the antigen of interest. The preferred height of the flow cell should be 50 μm or less (measured from the surface where the antigen of interest is provided, to the ceiling of the flow cell). Suitable flow cells are described, for example, in (WO 2008/132487). Other means for providing a flow-based analysis are also possible, however, such as the Octet (RTM) system Dip and Read assays from ForteBio/Sartorius.

In preferred embodiments, the flow-based analytical device comprises a mass-sensitive chemical sensor. Other analytical approaches are possible for the present invention (for example, through measurement of changes in capacitance, impedance, radioactivity, or fluorescence signals upon binding of components of the complex biological sample to the antigen of interest), but mass-sensitive chemical sensors can provide a convenient, label-free and highly sensitive means for carrying out the methods described herein.

In certain embodiments of the invention, the method is conducted in a label-free manner, by which it is meant that no additional chemical species (e.g. radioactive, particulate, magnetic, chromogenic, fluorescent, luminescent) is required to be attached to antibodies present in the sample in order for their binding to the antigen of interest to be detected. The presence of such labels invariably affects the kinetics of the interaction of antibodies present in a complex biological sample with the antigen, thereby leading to less accurate results. A labelling step also makes an analytical method more complex, time-consuming and expensive. All methods of the present invention may be conducted in a label-free manner. As explained above, an approach based on a mass-sensitive chemical sensor lends itself particularly well to such label-free methods.

A mass-sensitive chemical sensor can be defined as any device that allows for measurement of a property that scales proportionally to mass associated with or bound to a sensing surface of that device. Several such sensor techniques can potentially be used according to the invention, such as evanescent wave-based sensors, e.g. surface plasmon resonance (SPR, which is capable of registering mass changes by the associated change in refractive index at the surface), optical waveguides (also dependent on refractive index changes associated with mass binding events), optical diffraction, optical interference, ellipsometry and acoustic wave devices (for example quartz crystal microbalances (QCMs)). These sensor approaches are well established in the art (see, for example, Biomolecular Sensors, Gizeli and Lowe. Taylor and Francis, London; 2002). As explained above, complex biological samples present particular challenges in the use of evanescent wave, refractive index and/or optical sensors, due to the interference of other components of the sample with the measured signal, leading to potential inaccuracies in the measured kinetics of the antibody-antigen interactions. Accordingly, acoustic wave devices (such as QCMs) are particularly preferred for carrying out the present invention.

A QCM system utilizes the piezoelectric effect of a quartz crystal. In such a system a quartz crystal that is placed between two electrodes, which are connected to an AC-potential, begins to oscillate if the frequency of the AC-potential is close to the resonance frequency of the oscillation mode for the quartz crystal. The resonance frequency of the quartz crystal is a function of many parameters, such as temperature, pressure, cut angle of the crystal, mechanical stress and thickness of the crystal. The resonance frequency is inversely proportional to the thickness of the crystal.

Typical resonance frequencies used in liquid applications range from 1 MHz to 50 MHz. The crystal is normally AT-cut with a circular or square shape with a diameter of approximately 5-10 mm. The electrodes (driving and counter electrodes) are normally of gold on both sides, but other metals are not unusual. The electrodes are very thin compared to the quartz crystal plate and can therefore be considered as part of the crystal plate. When material is added to or removed from one of the electrodes, it becomes thicker or thinner, i.e. the associated weight of the electrode changes. As a consequence of the mass change of the electrode, the resonance frequency of the crystal plate will either decrease or increase and hence the change of resonance frequency can be measured to detect the mass change of the electrode. The mass resolution of a QCM system can be as low as 1 pg/cm², corresponding to less than 1% of a monolayer of hydrogen.

A typical QCM piezoelectric sensor instrument comprises a sensor element, a sample insertion unit, equipment for determining the piezoelectric properties (including the oscillation frequencies) of a quartz crystal, and signal presentation equipment and buffer and waste containers. According to the present invention, a complex biological sample is introduced into the sensor element by the sample insertion unit. The sensor element contains a piezoelectric resonator (the QCM sensor), a sample chamber, flow channels to and from the chamber and an oscillating circuit. The antigen of interest is immobilised (directly or indirectly) on the surface of the driving electrode, i.e that which is exposed to the contents of the flow channels. The complex biological sample induces an interaction with the antigen on the piezoelectric sensor surface, which can in turn be observed by monitoring the oscillating characteristics of the crystal plate, e.g. by measuring changes in the piezoelectric resonator frequency. The crystal plate is provided with electrical contact areas for the driving and counter electrodes on its surface, such contact areas being connectable to a signal source (e.g. an alternating voltage source) as well as to a measurement device.

Immobilisation of the antigen of interest on the sensor surface can be achieved by a variety of means, as would be appreciated by the skilled person. Common surface coating approaches include self-assembled monolayers (e.g. alkanethiols adsorbed onto gold) and/or polymeric matrices, each of which may bear functional groups which may be used for immobilising the antigen of interest or a larger species including the antigen of interest.

In line with the meaning of ‘antigen of interest’ explained above, it is also possible to study the interaction of antibodies in a complex biological sample with antigens of interest on the surface of cells immobilised on a sensor surface, as described in WO2010/106331. Such an approach can also be used with immobilised tissue, viruses, bacteria and other microorganisms.

In some embodiments, the complex biological sample is from a subject known or suspected to have been infected with, or who is carrying, a pathogen associated with the antigen, wherein the method allows for the immunological status of the subject to be assessed by means of the neutralising potential of the sample. The pathogen may be any virus or microorganism, or tumor cell, and it will be appreciated that the antigen of interest in this instance will be a known antigenic component of such a pathogen. Usefully, the method of the invention may be used to assess immunological status against a virus, such as a coronavirus, for example SARS-CoV-2 (in which case the antigen of interest may, for example, be the coronavirus spike protein).

In accordance with a second aspect of the invention, there is provided a method for assessing the immunogenic efficacy of a vaccine, the method comprising obtaining a complex biological sample from a subject previously inoculated with the vaccine, contacting the complex biological sample with a flow-based analytical device, the analytical device being provided with an antigenic component of the vaccine, allowing antibodies present in the complex biological sample to interact with the antigenic component, and determining the association-rate binding constant kₐ of the antibody-antigen interaction and/or the dissociation-rate binding constant k_{d} of the antibody-antigen interaction.

The immunogenic efficacy of a vaccine can be assessed according to the second aspect in an analogous manner to the assessment of the neutralising potential of a complex biological sample according to the first aspect. It will be appreciated that all embodiments of the first aspect of the invention may be applied to the second aspect. In particular, the assessment of kₐ and/or k_{d} and the additional assessment of one or more of the other parameters discussed above may usefully be employed in the second aspect in an analogous manner.

According to the second aspect, the kₐ and/or k_{d} (and optionally one or more of the other parameters discussed above) determined according to the method may be used as a factor in improving the antigenic design, formulation, administration and/or posology of the vaccine. The method of the second aspect allows for the rapid testing of subjects inoculated with a vaccine, and for the use of the parameters determined thereby to be compared with parameters measured in samples from subjects inoculated with e.g. different vaccine antigens (from the same infectious agent), different formulations of the same vaccine antigen, different adjuvant formulations of the same vaccine antigen, different administration routes and/or regimens, and/or different dosages of the same vaccine antigen. Such information can be used to inform modification of these aspects of the vaccine, the success of such modifications being indicated, according to the present invention, by improvements in the kₐ and/or k_{d} (and optionally one or more of the other parameters discussed above) of antibodies contained in complex biological samples obtained from subjects inoculated with the modified vaccines.

The invention thus also relates to an immunogenic composition or vaccine obtained or obtainable by a method in which the method of the second aspect is comprised.

In accordance with the first or second aspects, the results of carrying out the methods of these aspects may inform clinical decision-making in relation to a subject from whom the complex biological sample is obtained. Thus the invention may also include a method of altering the therapeutic or prophylactic treatment of the subject based on the results of these methods.

In a third aspect, the present invention also provides a method for assessing the neutralising potential of antibodies in a complex biological sample, the method comprising introducing the complex biological sample into a flow-based analytical device, the analytical device being provided with an antigen of interest, allowing antibodies present in the complex biological sample to interact with the antigen of interest, and determining the titre of specific antibody binding to the antigen of interest.

According to the third aspect, the titre of antigen-specific antibodies of all isotypes present in the complex biological sample is measured. This includes, for example, IgG and IgM antibodies. This measurement can be predictive of the neutralising potential of a complex biological sample, and can provide useful information in terms of the stage of an infection by a pathogen associated with the antigen of interest. In the early stage of an infection, IgM levels can be elevated, and this provides a higher titre in this measurement. In the later stages of an infection, IgG tends to dominate, with a reduction in titre in this measurement.

In a preferred embodiment of the third aspect, the ratio (titre of specific antibody binding to the antigen of interest): (titre of all soluble components of the complex biological sample binding to the antigen of interest) is also determined. This ratio (which uses the measured signal post-wash of non-specific components of the complex biological sample, compared to the measured signal pre-wash) is useful in predicting the neutralising potential of a complex biological sample.

In a fourth aspect, there is provided a method for assessing the neutralising potential of antibodies in a complex biological sample, the method comprising introducing the complex biological sample and a competing species into a flow-based analytical device, the analytical device being provided with an antigen of interest, allowing antibodies present in the complex biological sample, and the competing species, to interact with the antigen of interest, and determining the binding of specific antibodies from the complex biological sample to the antigen of interest.

In the method of the fourth aspect, a competition binding (or blocking) assay is carried out. The competing species is one or more species which are known to bind to the antigen of interest (e.g. in the case of the spike protein from SARS-COV2, the species may be ACE2 (angiotensin converting enzyme 2, the cell surface receptor the spike protein binds to in order to gain entry into cells). The competing species may be introduced into the analytical device prior to, simultaneously or following the introduction of the complex biological sample. In a preferred embodiment, the competing species is introduced prior to or approximately simultaneously with the complex biological sample. The binding of specific antibodies may be determined in terms of all isotype binding, or may be determined based on specific isotype(s) binding. For example, specific IgG binding may be determined by subsequently introducing an ant-IgG antibody. Complex biological samples having higher neutralising potential will show less reduction in specific antibody binding to the antigen of interest in the presence of the competing species than samples having lower neutralising potential. The association and/or dissociation rate constants of the antibody-antigen interaction may also be determined in the presence of the competing species. Complex biological samples having higher neutralising potential will show less observed deterioration in these kinetic parameters than samples having lower neutralising potential.

As would be appreciated by the skilled person, the embodiments of the first aspect described above may also be applied to the third and fourth aspects.

The present invention will now be described in more detail by way of example only, and with reference to the attached figures, of which:

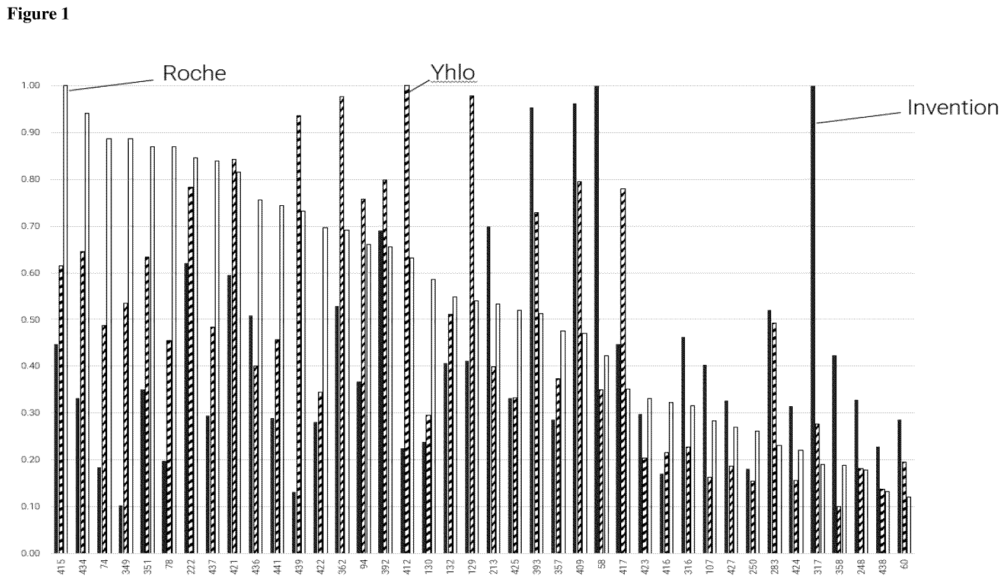

FIG. 1 shows the results of an assessment, using an Attana QCM-based assay, of the level of IgG antibodies against the receptor binding domain (RBD) of the spike protein of SARS-COV-2 in blood samples obtained from a set of subjects, wherein the highest IgG titre is given a score of 1.00, with the titre of the other samples being normalised against this (dark-fill bars; 1.00 ranking at subject 317). This is compared to results for the same set of subjects using two commercially available, serum incubation-based tests to determine IgG titre, where in each instance, the highest titre using the given method is given a score of 1.00 and the titre of the other samples using that method are normalised against this (Roche=non-filled bars, with 1.00 ranking bar at furthest left; Yhlo=hatched bars, with 1.00 ranking bar at subject 412);

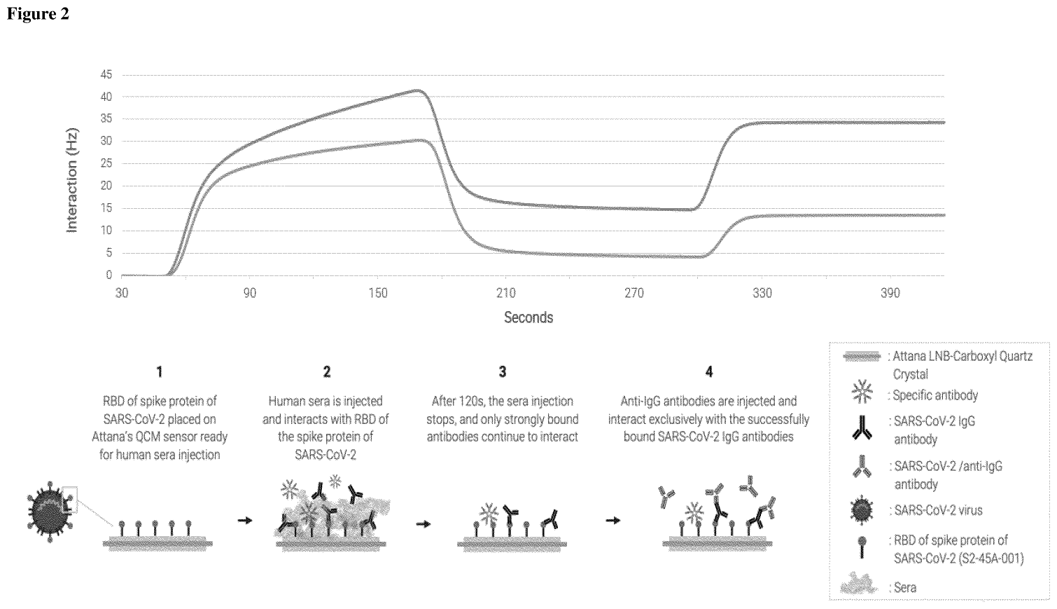

FIG. 2 shows overlaid QCM sensorgrams from the interaction of human capillary blood samples from two different subjects with a sensor surface to which the spike protein RBD of SARS-COV-2 was immobilised;

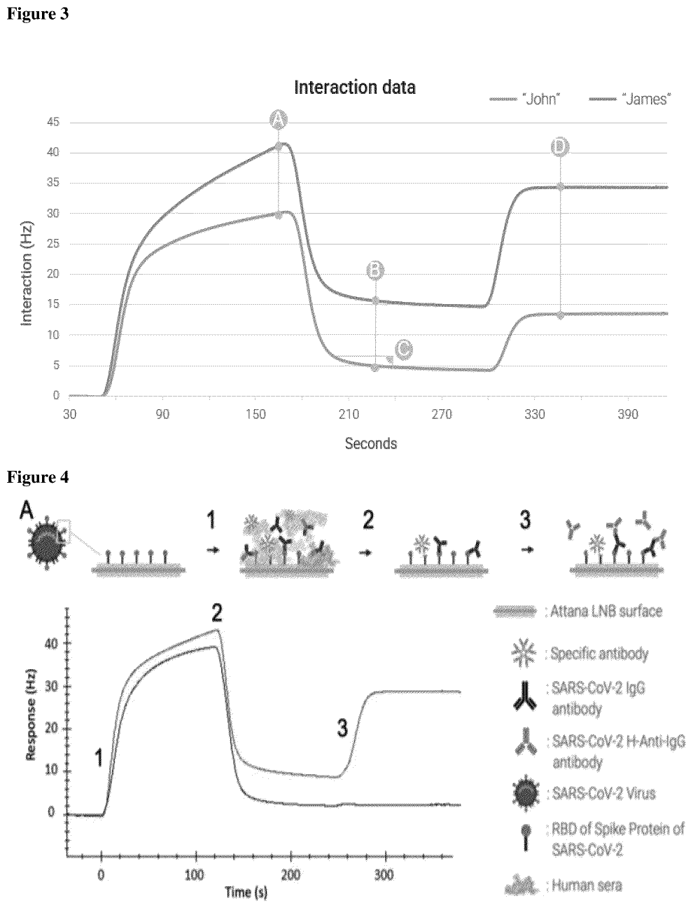

FIG. 3 shows overlaid QCM sensorgrams as in FIG. 2, with the differences in relevant kinetic and other parameters between the two blood samples highlighted;

FIG. 4 demonstrates another example of the AVA SARS-COV-2 IgG Immunoassay. (A) Schematic representation of assay procedure. RBD of the SARS-COV-2 spike protein is immobilized on an Attana low non-specific binding sensor chip. Human serum is injected (1) and allowed to interact with the immobilised RBD for 120 s, followed by buffer injection for 100 s (2) where only strongly bound antibodies continue to interact. Antihuman-IgG antibodies are injected (3) for the detection of bound IgG antibodies. The sensorgram displays the response in Hertz over time in relation to the events in panel A. The figure shows two samples with varying titers of SARS-COV-2 anti-IgG;

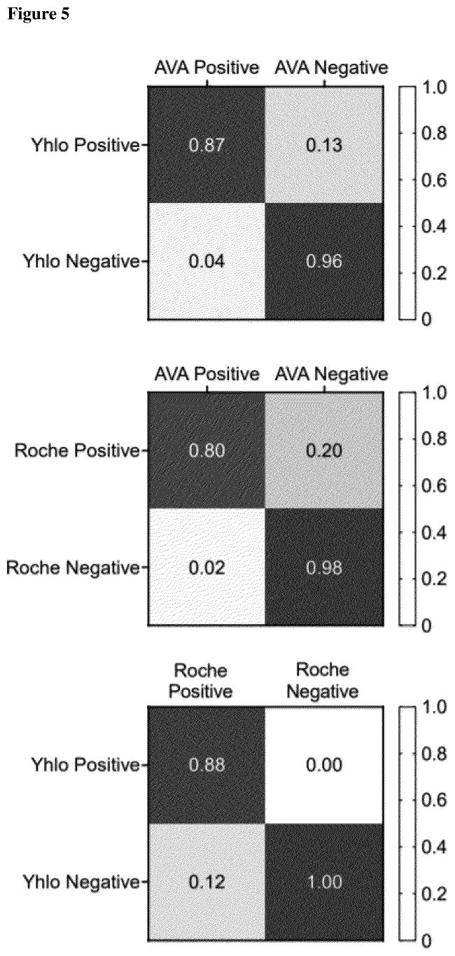

FIG. 5 summarises the coherence between AVA, Roche, and YHLO SARS-COV-2 antibody assays. Sera were analysed for the presence of antibodies against SARS-COV-2 RBD spike protein. Of the 119 analysed samples, 48, 52, and 59 were positive in the AVA, Roche, and YHLO assays. The figure represents coherence between AVA and YHLO (A), AVA and Roche (B), Roche and YHLO (C);

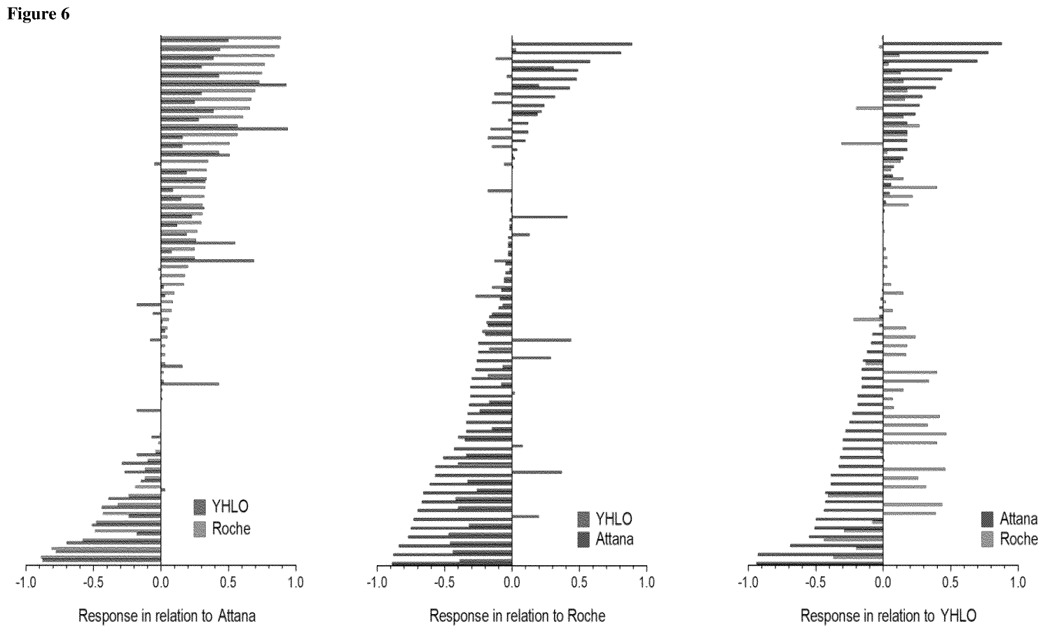

FIG. 6 shows Attana, YHLO, and Roche response-comparisons. The responses of all samples were normalised to the samples with the highest response in each assay and subtracted from the (A) AVA-, (B) Roche-, and (C) YHLO-response. The difference of the normalised response is plotted in each graph. In the graph ‘In relation to Attana’, each pair of bars represents Roche (upper bar in each pair) and YHLO (lower bar in each pair); in the graph ‘In relation to Roche’, each pair of bars represents YHLO (upper bar in each pair) and Attana (lower bar in each pair); and in the graph ‘In relation to YHLO’, each pair of bars represents Attana (upper bar in each pair) and Roche (lower bar in each pair);

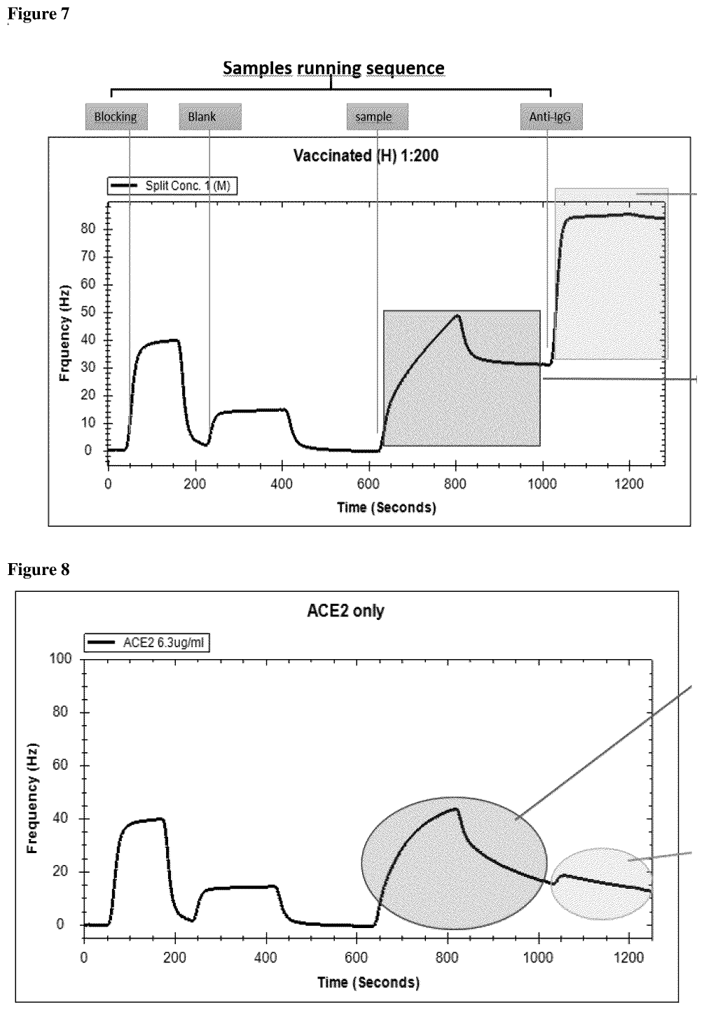

FIG. 7 shows a QCM sensorgram relating to the binding of antibodies from a serum sample from a Covid-19 vaccinated subject to a surface bearing immobilised SARS-COV2 RBD;

FIG. 8 shows a QCM sensorgram relating to the binding of ACE-2 to a surface bearing immobilised SARS-COV2 RBD;

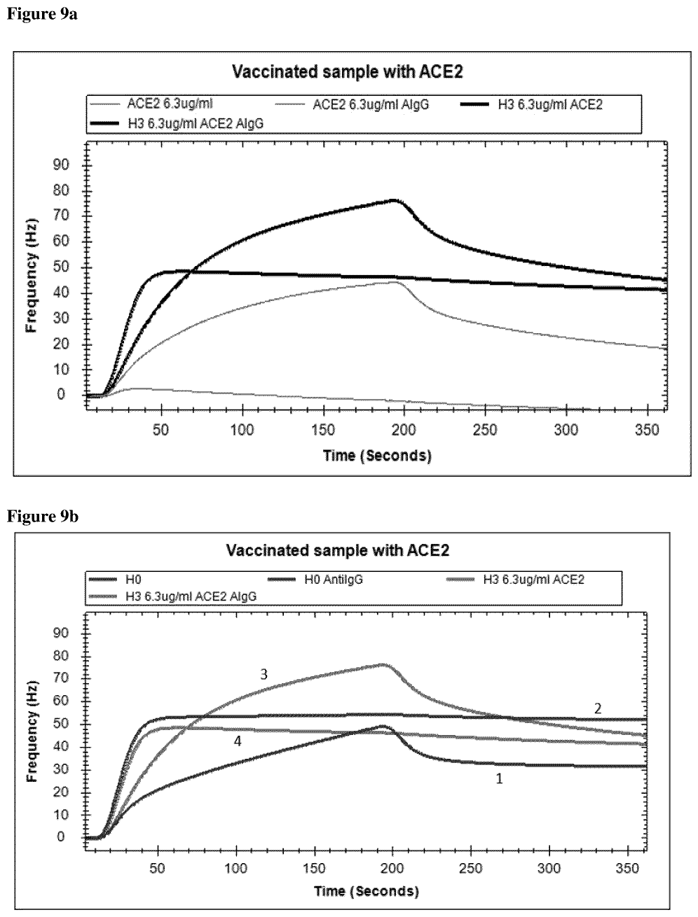

FIG. 9 shows (a) a QCM sensorgram relating to the binding of anti-IgG antibodies to the sensor surface following contact of a surface bearing immobilised SARS-COV2 RBD with ACE-2 only (lines 1 and 2 from the bottom), or following contact of a surface bearing immobilised SARS-COV2 RBD with ACE-2 simultaneously with a sample from a Covid-19 vaccinated subject (lines 3 and 4 from the bottom); and (b) a QCM sensorgram relating to the binding of anti-IgG antibodies to the sensor surface following contact of a surface bearing immobilised SARS-COV-2 RBD with a sample from a Covid-19 vaccinated subject (line 1 serum sample, line 2 anti-IgG), or following contact of a surface bearing immobilised SARS-CoV-2 RBD with ACE-2 simultaneously with a sample from a Covid-19 vaccinated subject (line 3 serum sample and ACE2, line 4 anti-IgG);

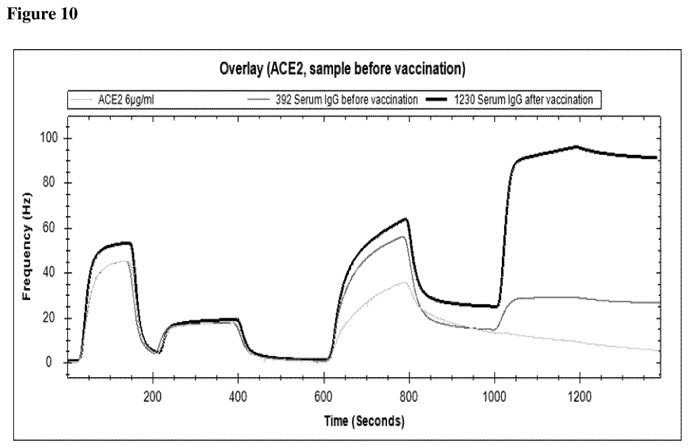

FIG. 10 shows a QCM sensorgram relating to the binding of antibodies from a serum sample from a Covid-19 vaccinated subject and non-vaccinated subject to a surface bearing immobilised SARS-COV2 RBD;

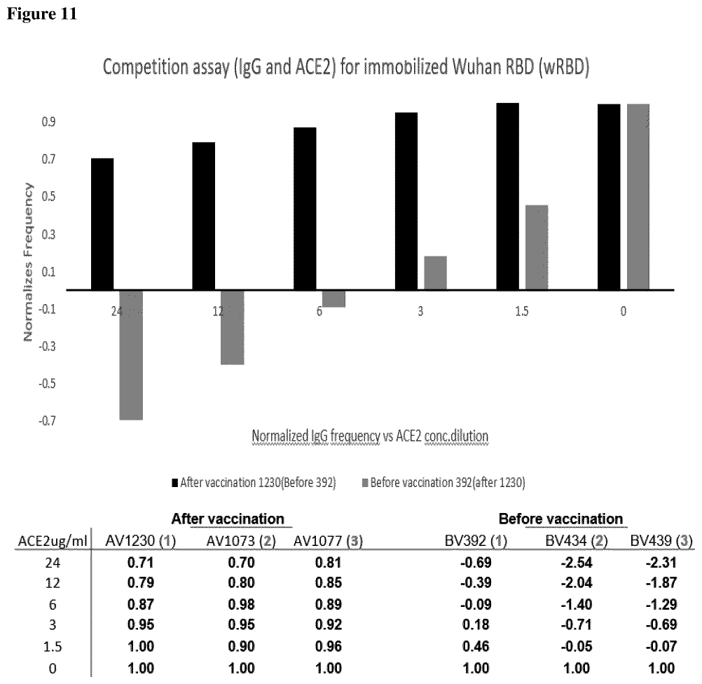

FIG. 11 shows the results of QCM-based blocking assays on serum samples from subjects before and after Covid-19 vaccination;

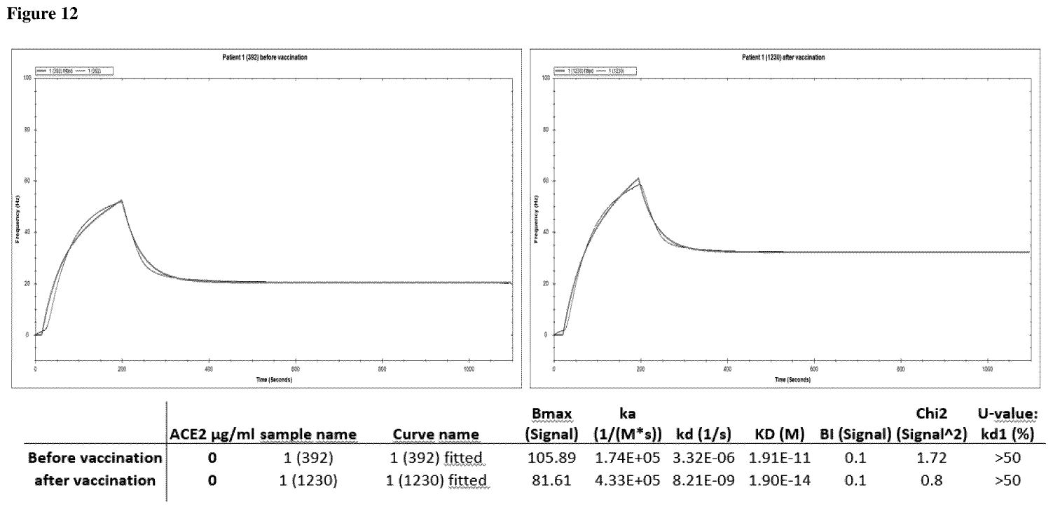

FIG. 12 shows the kinetic analysis of QCM sensorgram results relating to the binding of antibodies from a serum sample from a Covid-19 vaccinated subject and non-vaccinated subject to a surface bearing immobilised SARS-COV2 RBD;

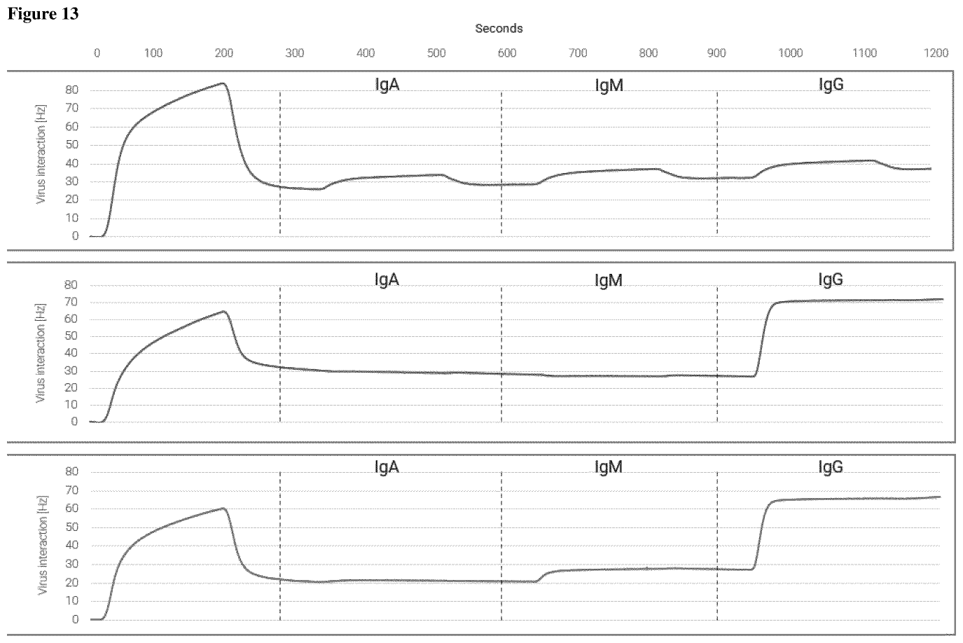

FIG. 13 shows QCM-based analysis of blood samples from a subject testing positive for Covid-19 at two different time points, and analysis of a blood sample from a family member of the subject; and

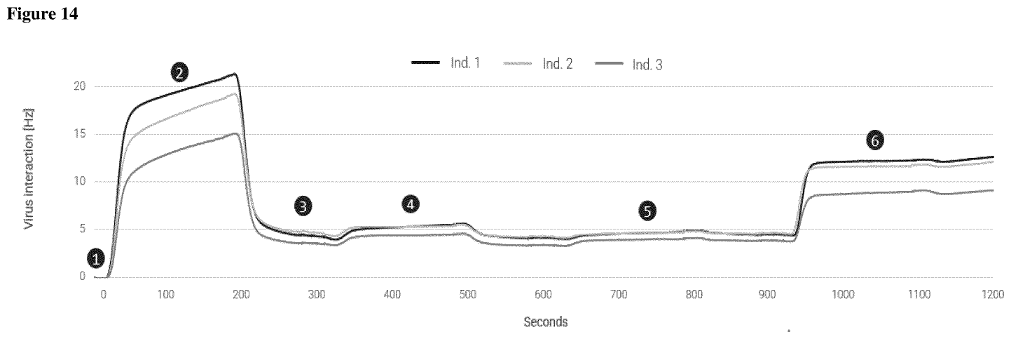

FIG. 14 shows QCM-based analysis of blood samples from subjects having received a tetanus vaccination.

### EXAMPLE 1

### Comparison of Assessment of Antibody Neutralising Potential Using Different Analytical Techniques

Blood samples were taken from 300 patients and tested for the presence of IgG antibodies against the spike protein of the SARS-COV-2 virus. In FIG. 1, each entry on the x-axis represents a different patient. For each patient, IgG was measured using the commercial, incubation-based, titre determination methods from Roche and Yhlo, and a QCM-based method. The patient sample showing the highest result for a given method was ranked as 1.00 (y-axis) for that method, and all other patient results for that method were normalised against this patient result. As can be seen, using the QCM-based approach, there are several patient samples showing high SARS-COV2 neutralising IgG capacity which are not ranked highly using the commercial methods. Equally, those patient samples which rank highest using the commercial methods do not rank consistently highly based on the QCM-based results.

### EXAMPLE 2

### Measurement of Anti-SARS-COV-2 Antibody Concentration and Kinetics in Blood Samples

FIG. 2 shows the results of two patients whose blood was tested for antibodies against SARS-COV2 using a QCM-based approach according to the invention. The different stages of the test are explained in the figure (bottom panel).

The immobilisation step (amine coupling) in this analysis was carried out in accordance with the following protocol:

### Material

3501-3001 Attana amine coupling kit (EDC (1-Ethyl-3-[3-dimethylaminopropyl]-carbodiimide hydrochloride) and sNHS (N-hydroxysulfosuccinimide) aliquots of 160 μL, prepared according to product protocol; 10 mM Sodium Acetate, pH 4.5; 1 M Ethanolamine, pH 8.5)
3506-3001 HBST 10×, diluted to 1×
3623-3103 Attana LNB Carboxyl sensor surfaces
RBD Spike protein of choice

### Preparations

1. Prime the system with running buffer (HBST 1×) and set the temperature to 22 deg C.
2. Dock two new LNB surfaces and start the flow at 100 μL/min for a minimum of 10 minutes.
3. When the baseline is without irregularities, lower the flow to 10 μL/min.
4. Wait until the baseline has stabilized at 2 Hz/10 min.

### Manual Immobilization Procedure

1. To properly save data from the experiment, remember to record the procedure by clicking on the green “play” button in the left corner of the software.
2. Mix thawed EDC 0.4 M and sNHS 0.1 M (1:1 dilution) prior to injection. Note: The mixed solution is not stable and should be used immediately.
3. Inject the EDC/sNHS-mix over channel A and channel B with the injection time set to 300 sec.
4. Dilute RBD Spike to 50 μg/ml in ligand immobilization buffer (10 mM sodium acetate, pH 4.5) to a volume of 250 μL.
5. Inject RBD Spike over channel A and channel B with the injection time set to 300 sec.
6. Inject ethanolamine (1 M, pH 8.5), over channel A and channel B with the injection time set to 300 sec.
7. Change buffer to proceed with experiments, or undock the surfaces and wrap them with parafilm for storage in a Falcon tube at 2-8 deg C.

### Automated immobilization Procedure

1. Open the list A200 Amine coupling identical.lst and edit the wells as appropriate. One empty well will be used for mixing of EDC/sNHS.
2. Dilute the RBD Spike to 50 μg/mL in ligand immobilization buffer (10 mM sodium acetate, pH 4.5) to a volume of 250 μL.
3. Set tray area to fill the wash trays and MTP correctly.
4. Run the list.
5. Change buffer to proceed with experiments, or undock the surfaces and wrap them with parafilm for storage in a Falcon tube at 2-8 deg C.

The QCM analysis (sometimes referred to herein as AVA-SARS-COV-2 IgG Immunoassay) was carried out in accordance with the following protocol:

### Material

Attana Cell™ 200 or Attana Cell™ 250 biosensor
Attana C-Fast software 3.0.0.2
3623-3103 Attana LNB Carboxyl sensor surfaces with amine coupled SARS-COV-2 RBD
Spike protein as per immobilisation protocol above
Blocking agent: Non-fat dry milk
Buffer: PBS/EDTA 10 mM
Regeneration: Glycine 10 mM pH 1
Anti-human IgG—Medix Biochemica monoclonal produced in mouse against human Fc fragment dissolved in 37 mM citrate, 125 mM phosphate, pH 6.0, 0.9% NaCl, 0.095% NaN3 as a preservative (Product #Anti-h IgG 7701 SPRN-5)
Serum Sample Quantity: 5 μl diluted 1:50 giving a total volume of 250 μl
Plasma Samples: Na-citrate, K-EDTA, Li-heparin

### Preparation

1. Prime the system with running PBS buffer and set the temperature to 22° C.
2. Dock two LNB surfaces with amine coupled SARS-COV-2 RBD and start the flow at 100 μL/min for a minimum of 10 minutes.
3. When the baseline is without irregularities, lower the flow to 10 μL/min.
4. Wait until the baseline has stabilized at 2 Hz/10 min.

### Instrument and Experimental Parameters

Temperature 22° C.
Flow rate: 10 μl/min
Blocking agent: Non-fat milk, 10 mg/ml
Running buffer: PBS
Regeneration: Glycine 10 mM pH 1
Injection time: 30 s
Plug volume: 10 μl
Serum dilution: 1:50 in running buffer+10 mM EDTA (can be adjusted if needed)

### Injection Sequence

Using the Attana C-Fast software:
   Select the wells in the deep well plate for the samples as appropriate
   Select the position for other blocking, blank and other reagents in the solvent tray
   Check the required running buffer or wash buffer
   Calculate the volume required for samples or reagents
   And run the samples sequence according to the following:
PBS running buffer at 10 μl/min stabilization
1 Blocking agent (non-fat milk, 2 mg/ml) in running buffer 30 s/50 s Assoc/Diss
2 Blank sample (PBS+10 mM EDTA)] 30 s/100 s Assoc/Diss
3 Serum sample 1 (diluted 1:50 in PBS+10 mM EDTA) 30 s/100 s Assoc/Diss
4 Anti-IgG 20 μg/mL 30 s/100 s Assoc/Diss
5 Regeneration (glycine 10 mM, pH 1) 75 s/wait 200 s
Repeat above steps for subsequent samples.

### EXAMPLE 3

### Differences in Antibody Neutralising Potential Between Blood Samples From Different Patients

FIG. 3 illustrates the various parameters which can be determined in accordance with the method of the invention. In the first part of the sensorgrams (up to label A), kₐ and the total (i.e. specific and non-specific) immune response may be determined. The next phase (through to label B) allows the concentration of specific antibodies (of all isotypes) to be determined (i.e. following stopping the injection of the blood sample at around 170 s on the x-axis, such that non-specifically bound components are removed by the buffer flow). Dissociation of specific antibodies (up to label C) allows k_{d} to be determined, and this reflects the ‘quality’ of the specific antibodies which remain bound after the washing step. Following introduction of an anti-IgG antibody, the titre of specific IgG antibody in the samples can be determined (label D).

### EXAMPLE 4

### Comparison of QCM-Based Analysis of Complex Biological Samples with ELISA-Based Immunoassays, and the Importance of Antibody-Antigen Binding Kinetics

### Introduction

The emergence of the SARS-COV-2 virus and the subsequent COVID-19 pandemic has driven the need to quickly establish rapid, sensitive, and robust diagnostic tools for informing clinicians and policymakers and for use in vaccine development. Antigen-tests to determine the presence of the virus in individuals and in the environment, together with serological tests to assess the immune response after exposure to the virus or vaccination, have been tools central for guiding society's response to the pandemic. These methods have contributed directly to patient care and underlying pathogenesis, our understanding of SARS-COV-2 ecology and evolution, assessments of its spread and mutational frequency and the impact of measures used to limit the spread of the virus, e.g., by vaccination.

Diagnostics providing insight concerning individual immune responses to SARS-COV-2 infection by quantifying circulating antibodies are important in both the clinical context and for evaluating the broader impact of vaccination programs and mapping the spread of the virus. Necessity has driven the parallel establishment of commercialised diagnostic strategies, where a variety of detection principles has been used. Most commonly, detection is founded upon an antibody targeting ELISA-based method, as exemplified by the electrochemiluminescence assay (ECLIA) Elecsys® Anti-SARS-COV-2 serology test kit developed by Roche, and YHLO's chemiluminescence immunoassay (CLIA). Both assays require initial patient serum incubation with SARS-COV-2 antigens before a detection step giving an endpoint measurement. In contrast, Attana's AVA-immunoassay is a label-free flow-based system that uses quartz crystal microbalance (QCM) methodology to detect antibody-binding in real-time, providing insights into the kinetics of the antibody-antigen interaction. The Roche diagnostic platform employs the nucleocapsid (N)-antigen, the YHLO assay a combination of N-and spike protein(S)-antigens, and the Attana platform the S-antigen for antibody capture.

The objective of the present study was to investigate how these three platforms based upon different detection techniques and antibody targets perform relative to one another.

### Materials and Methods

### Blood Samples:

Initially, serum samples (n=335) were collected for the assessment of seroprevalence in healthcare workers in Kalmar County, Sweden. None of the donors had been vaccinated against SARS-COV-2. All samples were screened for SARS-COV-2 antibodies using the Roche ECLIA-based assay. From the 335 samples, 59 samples tested positive and 276 negative. All positive samples (n=59) were together with 60 randomly selected negative samples subjected for the YHLO-and AVA-assays.

### ROCHE—Electrochemiluminescence Assay (ECLIA):

Serum samples were evaluated for antibodies using the Elecsys® Anti-SARS-COV-2 serology test kit with the Roche Electro-chemiluminescence Assay (ECLIA) platform. The assay is designed to measure total antibodies (IgA, IgM, and IgG) against the N-antigen of SARS-COV-2 in human serum or plasma. The assay was performed according to the manufacturer's protocol (2020 edition). Briefly, serum was incubated for 9 min with biotinylated and ruthenylated nucleocapsid antigens, followed by incubation for 9 min with streptavidin-coated magnetic microparticles. The microparticles were captured on the surface of the electrode by magnetic force. Then, unbound substances were removed, and the electrochemiluminescence was induced by applying a voltage that produces the signal proportional to the amount of antibodies present in the serum sample. The cut-off values were pre-defined by the manufacturer, ≥1.0 U/mL were considered as positive and those <1.0 U/mL as negative.

### YHLO Chemiluminescence Immunoassay (CLIA):

Serum samples were tested using the YHLO iFlash-SARS-COV-2 IgG with YHLO's iFlash immunoassay analyzer. The assay is designed for quantitative measurement of IgG antibodies against a combination of N- and S-antigens of SARS-COV-2 in human serum or plasma. The assay was performed according to the manufacturer's prescribed protocol (2019 edition). Briefly, serum was incubated with SARS-COV-2 antigen-coated paramagnetic microparticles, followed by the addition of acridinium-labeled anti-human IgG antibody, which produces a light signal directly proportional to the number of antibodies detected in the sample. The total assay time was 45 min and the cut-5 off values were pre-defined by the manufacturer, ≥10 U/mL were considered as positive and those <10 U/mL as negative

### Attana Virus Analytics SARS-COV-2 IgG Immunoassay (AVA):

The serum samples were tested for anti-SARS-COV-2 antibodies using the AVA™ SARS-CoV-2 IgG Immunoassay kit and Attana Cell™ 200 biosensor (see FIG. 4). The assay is designed to quantitatively measure IgG antibodies against S-antigens of SARS-COV-2 in human serum or plasma. The receptor binding domain (RBD) of the SARS-COV-2 S-antigen was immobilized onto a low non-specific binding (LNB) sensor chip using amine coupling technique according to the manufacturer's protocol (see above). A 20 μl serum sample was injected at a flow rate of 10 μl/min interacting with the target for 120 s under continuous flow followed by buffer for 100 s. An anti-human-IgG antibody was subsequently injected for 30 s, followed by buffer for 100 s, and the change in frequency was recorded for a total of 120 s from the injection of the anti-human-IgG antibody. The signal was recorded as the mass change of the sensor surface, which correlates to the amount of IgG antibodies present in the sample. Linearity tests were performed on five randomly selected positive samples diluted in a negative serum pool, and PBS. A reproducibility test was performed by evaluating the response of ten undiluted randomly selected samples at two consecutive days on two individual chips. The total assay time was 8 min and cut-off values were pre-defined by the manufacturer, ≥2.5 Hz were considered as positive and those <2.5 Hz as negative

### Results and Discussion

The CE-marked ECLIA-based Roche, CLIA-based YHLO, and QCM-based Attana platforms are each based upon different physical principles for detection of individual immune response to the SARSCOV-2 virus spike protein, or to vaccination. The ELISA-based Roche (Roche 2020) and YHLO assays have been previously reported. The QCM-based Attana AVA platform (FIG. 4) was here validated for linearity and reproducibility. Five randomly selected positive samples were serially diluted in serum or buffer. The observed response in two-fold dilutions up to 1:64 was compared to the theoretically expected response, expressed as the response in undiluted serum related to the dilution factor. All five samples showed a low deviation between the observed and expected response, the correlation factors for both dilution in serum and buffer samples were r2-0.96, indicating a good robustness for sample dilution in both serum and buffer. The reproducibility was tested on two consecutive days on two different chips on ten randomly selected samples. The average response difference was calculated to be 5.3%.

In addition to physical principles for detection, the platforms also differ through their targeting of either one or both of the N- and S-antigens, the impact of which was deemed a parameter important to consider in the comparison of data arising from the three platforms. To compare these platforms, the cross-platform comparative study was undertaken. From an initial series of 335 donor samples analysed on the Roche-derived ECLIA assay, all 59 positive, and 60 randomly selected negative samples were then subjected to the CLIA-based YHLO, and Attana AVA assays.

From the collective data, comparable numbers of positive IgG responses were observed with the Attana (48), Yhlo (52), and Roche (59) assays. An initial cross-comparison of data revealed an overall good agreement between the three methods (FIG. 5). In summary, of the 52 samples positive in the case of the YHLO assay, 45 were positive in the AVA assay and 52 in the Roche assay. Of the 59 samples positive in the Roche assay, 47 were positive in the AVA assay and 52 in the YHLO assay. The corresponding numbers for the 67 negative samples in the YHLO assay were 64 in the AVA assay and 60 for the Roche. Of the 60 negative samples in the Roche assay, 59 were also negative in the AVA assay and 60 when interrogated using the YHLO platform.

To compare the inter-assay response of the positive samples, all samples positive in each assay were normalized against the sample that gave the highest response within each of the three assays (AVA, n=48; YHLO, n=52; Roche, n=59, FIG. 6). The normalised response was subtracted from that of the assay it is compared with. The positive population shows a significant divergence where some samples align well in all assays, and others differ substantially.

As assay response depends on the concentration and binding affinity of the antibodies towards their target antigen. The label-free AVA assay favours antibodies with higher affinity since the interaction time with its immobilised antigen is shorter and observed in a continuous flow, which also allows the assay to be used to provide access to kinetics data (Brien et al. 2013, above). Accordingly, a low antibody titer will result in a low response in the Roche and YHLO assays, yet if these antibodies have a good kinetic profile, the AVA response will be relatively higher, and vice versa.

### Conclusions

The COVID-19 pandemic has driven the development of new diagnostics for determining individual immune responses to infection by variants of the SARS-COV-2 virus and by vaccination. The ECLIA-based Roche, CLIA-based YHLO, and QCM-based Attana platforms have here been compared with respect to their performance when challenged with positive and negative samples derived from a cohort of 119 donors. Despite being based upon different physical principles and using different configurations of the N-and S-antigens, the degree of variation between the three platforms over the samples studied was comparable. The inherent high sensitivity of (electro) chemiluminescence, as deployed in the YHLO CLIA and Roche ECLIA platforms, could provide access to even lower levels of antibody-antigen interaction detection. However, the label-free flow system QCM-based Attana platform can provide direct access to antibody-antigen interaction kinetics data due to the measurement of the antibody-antigen interaction being performed in real time. Access to such data also opens up its use in vaccine screening/development.

### EXAMPLE 5

### QCM-Based Analysis of Serum Antibody Binding to Immobilised SARS-COV-2 Spike Protein RBD Under Blocking Conditions With ACE-2 and

### Determination of Kinetic Parameters of Antibody-Antigen Interaction

An aim of this experiment was to validate the neutralisation effect of ACE2 and how it competes with the covid 19 antibodies to bind to the RBD. This allows for quantitative analysis and comparison of antibodies from blood samples before and after vaccination, and by means of the flow-based analysis, determination of the binding kinetics.

### Method

50 μg/ml of RBD (receptor binding domain) was immobilised on LNB Carboxyl chips as described above, to test the competition between the human IgG in serum samples and ACE2 to bind to the immobilized SARS-COV-2 RBD. Samples were selected from Covid-19 vaccinated donors and due to higher frequency of antibodies in those samples we diluted the samples with the dilution 1:200. The ACE2 was mixed with the samples in the following dilution series: 100 μg/ml, 25 μg/ml, 6.3 μg/ml, 1.3 μg/ml, 0.4 μg/ml and 0.0 μg/ml which is the sample without ACE2 (H) diluted 1:200.

Immobilization: the immobilization followed the attana protocol (Amine coupling of RBD Spike on LNB Carboxyl, as described above), where 50 μg/ml RBD was immobilized on LNB Carboxyl chips with a yield of around 60 Hz.

For sample analyses we followed the Attana protocol (AVA SARS-COV-2 IgG Immunoassay, as described above), with some modification. Patient samples were collected from vaccinated donors and from cohort study from Region Kalmar. Before using the samples from the cohort study we tested the system using the vaccinated donor samples and due to high antibody frequency, we diluted the samples with the dilution 1:200 instead of 1:50, and increased the contact time from 120 seconds to 180 seconds for blanks and samples. FIG. 7 shows the full sample analysis steps which first start by blocking the unreacted functional groups on the carboxyl surface, 2^{nd} the blank, which has the same contact and dissociation time as the sample, then 3^{rd} followed by the serum sample containing antibodies, 4^{th} the anti-IgG, then finally 5^{th} the regeneration phase (during which dissociation of the antibody-antigen complex can be monitored).

The data shows a high frequency shift for the donor vaccinated samples, implying a high specific antibody response (first box from left), which is confirmed to be high in specific IgG by means of the high shift when anti-IgG is introduced (second box).

As a control experiment (FIG. 8), we injected only ACE2 (6.3 μg/ml) and we can see the full sample analyses steps which first start by blocking, 2^{nd} the blank, which has the same contact and dissociation time as the sample, then 3^{rd} the ACE2 sample, 4^{th} the anti-IgG, then finally 5^{th} the regeneration.

The data shows the continuous decrease in frequency shift due to dissociation after the contact time for the ACE2 (first circle from left), and due to the lack of response when Anti-IgG is introduced, confirms that anti-IgG does not bind to the ACE2 (second circle).

A dilution series of ACE2 was injected together with the serum sample. It was found that the overall frequency shift for each sample was increased by increasing the ACE2 concentration. This is due to the greater binding of ACE2 to the RBD surface as ACE2 concentration is increased in the sample. Hence the importance of ACE2 only and serum sample only (i.e. no ACE2) control experiments.

In contrast, when ant-IgG is introduced after the ACE2/serum samples, it is observed that the frequency shift for each sample due to the anti-IgG binding decreased slightly by increasing the ACE2 concentration. This is due to competition for the RBD binding sites on the surface, by which the presence of ACE2 reduces the amount of serum antibody (IgG) which binds to the RBD.

In FIG. 9a, there is compared the frequency shifts for ACE2 (6.3 μg/ml) binding to immobilized SARS-COV-2 RBD (second line from the bottom), followed by the frequency shift for anti-IgG injection (bottom line), with the frequency shift for antibody binding from a vaccinated patient serum sample with 6.3 μg/ml ACE2 (top line), followed by the frequency shift for anti-IgG injection (second line from top)). Similarly, FIG. 9b shows a complementary set of experiments, in which the ACE2-only injection is replaced with a vaccinated patient serum sample (with no ACE2) injection.

Based on the analysis of multiple patient samples in this way, it was concluded that although the ACE2 decreased the binding of sample IgG to the RBD surface, even with the highest concentration of ACE2 tested this effect was not dramatic in the vaccinated patients tested.

This method was then applied on some selected patients' samples (before and after vaccination) from the region Kalmar cohort study. Herein the data is presented for one patient sample collected before vaccination (392) and after vaccination (1230)

In this experiment 50 μg/ml of RBD was immobilized on LNB Carboxyl chips to test the competition between the human IgG and ACE2 to bind to the immobilized RBD. The serum samples were diluted 1:50, and we increased the contact time from 120 seconds to 180 sec for the blanks and the samples. The ACE2 was mixed with the samples in the following dilution series: 24 μg/ml, 6 μg/ml, 1.5 μg/ml and 0.0 μg/ml which is the sample without ACE2 diluted 1:50.

FIG. 10 shows the full sample analyses steps which start first by blocking, 2^{nd} the blank, which has the same contact and dissociation time as the sample, then 3^{rd} the patient sample or ACE2 sample, 4^{th} the anti-IgG then finally 5^{th} the regeneration.

The data shows a higher frequency for the patient sample ((1) 1230, top line) after vaccination, followed by a higher shirt when anti-IgG is introduced, implying a significant increase in specific IgG against Covid-19 after vaccination. This is compared to the patient sample ((1) 392) before vaccination with lower frequency shifts (middle line). The ACE2-only sample is shown in the bottom line.

By studying this system using the dilution series of ACE2, it was possible to conclude that the ACE2 did not affect the ability of IgG in vaccinated samples to bind to immobilised RBD to a great extent, and also that the IgG in the patient samples exhibited faster and stronger binding to RBD than did ACE2.

By normalising the data to 1.0 to compare the samples before and after vaccination and the effect of ACE2 in both conditions, it can also be seen the extent to which the ACE2 affected the IgG for samples before vaccination compared to samples after vaccination. In FIG. 11, the immobilised IgG from the serum samples (as determined using the subsequent anti-IgG response) is shown in this normalised manner for each ACE2 concentration (at each concentration, the before vaccination sample is on the right, the after-vaccination sample on the left). Note the difference in particular at 1.5 μg/ml ACE2 compared to that at 0 μg/ml ACE2 when comparing the samples before and after vaccination. The anti-IgG normalised frequency shift date for three separate patients is shown in the table in FIG. 11.

### Determination of Kinetic Parameters of Antibody-Antigen Interaction

Using pre-vaccination and post-vaccination samples as described above, the kinetic parameters of the interaction between antibodies in the samples and immobilised SARS-COV-2 RBD were determined using the QCM-based methods described above, and employing an approach as described by Brien et al. (2013, see above). The molar concentration of antibody in the samples was estimated by assuming an antibody molecular weight of 150 kDa in each instance, and the data was fitted to a kinetic model using the Attester Evaluation software from Attana AB. In order to determine the dissociation rates, which for certain samples were very slow (i.e. low k_{d}), highly stable experimental conditions were maintained, to ensure that high sensitivity and high signal: noise was obtained.

FIG. 12 shows the results for a pre-vaccination sample (left) and post-vaccination sample (right). The determined kinetic parameters are shown in the table. It can be seen that following vaccination, the antibodies in this sample exhibited an association constant kₐ around 2.5× higher than those in the sample pre-vaccination. The dissociation constant was determined over an extended period (900 seconds) to gather further data on high affinity antibodies, and it is shown that the antibodies in the post-vaccination sample exhibited a dissociation constant k_{d} around 400× lower than those in the sample pre-vaccination.

In analysis of other pre-and post-vaccinated samples by the same method, it was found in one comparison that kₐ increased from 5.05×10⁴ to 7.16×10⁵ (an increase of 14×), and k_{d} decreased from 1.04×10⁻⁹ to 8.71×10⁻¹⁷ (a decrease of around 10⁷×); and in another comparison that kₐ increased from 2.23×10⁵ to 4.27×10⁵ (an increase of approximately 2×), and k_{d} decreased from 2.41×10⁻¹¹ to 1.78×10⁻¹⁵ (a decrease of around 10⁵×).

These results indicate a strong, high-quality neutralising capacity in the post-vaccinated patient samples compared to the pre-vaccinated samples. The increase in kₐ and decrease in k_{d}, both of which are determined in real-time in a complex biological sample, correlate with the improved immunological status of the vaccinated patient, and hence the neutralising capability or capacity of the sample obtained therefrom. Such a correlation has not been recognised in the prior art, where mere presence/absence of antibodies or semi-quantitative antibody titre determinations prevail, and which are performed in media which make accurate kinetics determinations problematic in any event.

The methods of the present invention provide results which correlate with functional, clinical immunological status. Blood samples were taken from subjects, all of whom were currently testing positive for SARS-COV2 (based on lateral flow and/or PCR testing). Subjects were divided into those reporting no (or mild) symptoms of Covid-19, and those reporting clinically meaningful symptoms (e.g. cough, fatigue, loss of taste and/or smell). Determination of serum antibody binding kinetics to immobilised SARS-COV2 RBD was determined using a QCM biosensor, as described above. It was found that there was a strong correlation between high kₐ and the presence of no (or mild) symptoms, and between low kₐ and the presence of clinical symptoms of Covid-19. The lateral flow and PCR testing results were completely unable to discriminate between subjects in this way.

### EXAMPLE 6

### QCM-Based Analysis of Serum Antibody Binding to Immobilised SARS-COV-2 Spike Protein RBD: Different Time Points and Different Subjects

FIG. 13 shows QCM-based analysis of blood samples from a subject testing positive for Covid-19 (by lateral flow and PCR testing), using a sensor surface bearing the SARS-Cov-2 RBD. The top sensorgram shows the results from a sample taken shortly after Covid-19 testing confirmed the presence of an infection. Phase 1 of the sensorgram shows the initial attachment of all specific and non-specific binding components to the antigen, followed by the removal of the serum flow and the consequential dissociation of non-specifically bound and weakly interacting components. Subsequently, the surface was treated with anti-IgA, anti-IgM and anti-IgG, respectively. It can be seen that each of these antibody isotypes was present in the sample, and each isotype exhibited specific binding to the antigen.

The middle sensorgram relates to a sample from the same individual taken 9 days later. The ‘maturation’ of the serum immune response can be seen, in that IgA and IgM are essentially absent in the specific binding response, whereas IgG is present in a high titre.

The lower sensorgram relates to a sample taken from a co-habiting family member of the subject of the upper and middle sensorgrams. This sample was taken on the same day as that used in the middle sensorgram. It can be seen that the family member's immune response is also relatively mature, with a strong IgG response. Some IgM response is still present, however, suggesting that maturation of the immune response is still taking place.

It can also be seen that the shape of the association phase differs from the first test to the one from the same subject taken 9 days later. One reason for this is that there are no (or negligible) IgA and IgM in the 9 days later sample to compete with the IgG binding, so the shape of the association phase is more due to IgG in the latter sample. This indicates that the IgG binding in the day one sample is not significantly faster than the IgA or IgM binding. For a high neutralizing effect it is desirable that the IgG are significantly faster than other species.

### EXAMPLE 7

### QCM-Based Analysis of Serum Antibody Binding to Immobilised Tetanus Toxoid in Tetanus-Vaccinated Subjects

Blood samples were taken from three subjects (Individuals (Ind) 1, 2 and 3), each of whom was previously vaccinated against tetanus and showed no symptoms of disease. The blood samples were diluted with buffer and introduced into an Attana QCM biosensor having a surface to which was immobilised the tetanus toxoid protein. In FIG. 14, this is represented by stage 1, i.e. at a resonant frequency shift of zero before introduction of the samples, and stage 2, when the diluted blood samples are introduced and components of the blood interact with the immobilised toxoid protein.

After 180 s, the sample injections were stopped, and weakly bound and non-specifically bound components dissociate from the sensor surface (stage 3). In stages 4, 5 and 6, anti-IgA, anti-IgM and anti-IgG antibodies, respectively, are introduced to assess the type of the serum immunological reaction. It can be seen that in all three subjects, the immunological reaction is relatively mature, with IgG predominant. In addition, all three subjects display very high kₐ during stage 2, indicating a high neutralising capacity for tetanus. This is in agreement with the clinical immunological status of all three individuals. Calculated kₐ values from these results were all greater than 10⁶ M⁻¹s⁻¹.

The foregoing Examples are intended to illustrate specific embodiments of the present invention and are not intended to limit the scope thereof, the scope being defined by the appended claims. All documents cited herein are incorporated herein by reference in their entirety.

1. A method for assessing the neutralising potential of antibodies in a complex biological sample, the method comprising contacting the complex biological sample with a flow-based analytical device, the analytical device being provided with an antigen of interest, allowing antibodies present in the complex biological sample to interact with the antigen of interest, and determining the association-rate binding constant kₐ of the antibody-antigen interaction and/or the dissociation-rate binding constant k_{d} of the antibody-antigen interaction.

2. The method of claim 1, wherein the binding of all soluble components of the complex biological sample to the antigen of interest is also determined.

3. The method of claim 1 or claim 2, wherein the titre of specific antibody binding to the antigen of interest is also determined.

4. The method of any preceding claim, wherein the association-rate binding constant kₐ is determined, and preferably the dissociation-rate binding constant k_{d} is also determined.

5. The method of any preceding claim, wherein the titre of specific IgG binding to the antigen of interest is also determined.

6. The method of any preceding claim, wherein two or more of kₐ, specific antibody titre, k_{d}, and specific IgG titre are determined using the same analytical device.

7. The method of any preceding claim, wherein the complex biological sample is selected from blood, serum, plasma, saliva, sputum, biopsy eluate and lavage liquid.

8. The method of claim 7, wherein the complex biological sample is selected from blood, serum and plasma.

9. The method of any preceding claim, wherein the complex biological sample is diluted with a buffer.

10. The method of any preceding claim, wherein kₐ is determined over a time period which ends prior to the maximum level of antibody-antigen interaction being reached, such as the time period which ends at the point where 50% of the maximum level of antibody-antigen interaction is reached.

11. The method of claim 1 or claim 4, wherein kₐ and/or k_{d} of the antibody-antigen interaction are compared with the equivalent rate constant(s) for the interaction between antibodies in a control complex biological sample from a subject demonstrating clinically significant immunity against a pathogen associated with the antigen, or are compared with the equivalent rate constant(s) for the interaction between one or more commercially available therapeutic antibodies and their respective biological target antigens.

12. The method of any preceding claim, wherein the flow-based analytical device is provided with a flow cell, the antigen of interest being immobilised in the flow cell and the complex biological sample being introduced into the flow cell for the purposes of the antibody-antigen interaction.

13. The method of any preceding claim, wherein the flow-based analytical device comprises a mass-sensitive chemical sensor.

14. The method of claim 13, wherein the chemical sensor comprises a quartz crystal microbalance.

15. The method of any preceding claim, wherein the measurement of the interaction between antibodies present in the complex biological sample and the antigen of interest is performed in a label-free manner.

16. The method of any preceding claim, wherein the complex biological sample is from a subject known or suspected to have been infected with a pathogen associated with the antigen, and wherein the method allows for the immunological status of the subject to be assessed.

17. A method for assessing the immunogenic efficacy of a vaccine,, the method comprising obtaining a complex biological sample from a subject previously inoculated with the vaccine, contacting the complex biological sample with a flow-based analytical device, the analytical device being provided with an antigenic component of the vaccine, allowing antibodies present in the complex biological sample to interact with the antigenic component, and determining the association-rate binding constant kₐ of the antibody-antigen interaction and/or the dissociation-rate binding constant k_{d} of the antibody-antigen interaction.

18. The method of claim 17, wherein the kₐ and/or k_{d} determined according to the method is used as a factor in improving the antigenic design, formulation, administration and/or posology of the vaccine.

19. A method for assessing the neutralising potential of antibodies in a complex biological sample, the method comprising contacting the complex biological sample with a flow-based analytical device, the analytical device being provided with an antigen of interest, allowing antibodies present in the complex biological sample to interact with the antigen of interest, and determining the titre of specific antibody binding to the antigen of interest.

20. The method of claim 19, wherein the ratio (titre of specific antibody binding to the antigen of interest):(titre of all soluble components of the complex biological sample binding to the antigen of interest) is also determined.

21. A method for assessing the neutralising potential of antibodies in a complex biological sample, the method comprising contacting the complex biological sample and a competing species with a flow-based analytical device, the analytical device being provided with an antigen of interest, allowing antibodies present in the complex biological sample, and the competing species, to interact with the antigen of interest, and determining the binding of specific antibodies from the complex biological sample to the antigen of interest.
